(19) United States
(12) Patent Application Publication
(10) Pub. No.: US 2007/0244500 A1
(43) Pub. Date: Oct. 18, 2007

(54) Percutaneous introducer and associated balloon catheter

(75) Inventors: Daniel R. Lucas, Anaheim, CA (US); Celia L. Lucas, Anaheim, CA (US)

Correspondence Address: WILLIE KRAWITZ, 3001 CHAPEL HILL ROAD, ORANGE, CA, 92687-1927, US

(73) Assignee: 422 MEDICAL LABORATORIES

(21) Appl. No.: 11/391,094

(22) Filed: Mar. 27, 2006

(51) Int. Cl. A61M 5/178; A61M 29/00

(52) U.S. Cl. 606/194; 604/167.01

(57) ABSTRACT

A balloon catheter construction for percutaneous use is provided employing a balloon polymer material bonded to an introducer shaft with a bonding agent comprising a low molecular weight oligomer corresponding to the higher molecular weight polymer of the balloon. The oligomer functions alone or together with a primer which enables bonding to the shaft during heat curing, thereby securing the balloon thereon. The balloon introducer catheter is designed to effectively block emboli material which are released from say a plaque rupture, or other site and to then to remove such material by suction from a blood vessel such as a vein or artery.

### BACKGROUND OF THE INVENTION

This invention relates to a new and improved percutaneous introducer balloon catheter providing a stable, balloon with suitable elasticity during use and a reduced possibility of rupturing which could result in a rupturing of an artery. This invention also relates to a balloon catheter, which in conjunction with suction and/or filtering, can effectively remove emboli or other material from a plaque rupture or other site and thereby prevent such material from entering into a patient's blood stream.

A typical balloon catheter for use in a system which is designed to remove debris and blood particles from a vein or artery by suction is described in Japanese Patent Application Serial No.: 2004-134003, filed Apr. 28, 2004 in Japan by the inventor Hiroki Horita, and incorporated by reference herein. While the overall system itself is useful, Horita fails to describe a balloon portion having suitable elastic, compliant and bonding properties to the introducer shaft that would in fact prevent the escape of particles and prevent rupture and damage to vessels.

Obviously, it would be desirable to provide a balloon catheter which can effectively block the escape of blood particles and emboli materials arising from say dilation of a stenosis lesion and also permit employing the balloon catheter in conjunction with a suction device.

In addition, it would be desirable to provide a balloon catheter with sufficient elastic properties to provide effective expansion without causing blow-out of a vein or artery, and could provide suitable low friction properties to enable easy insertion and retraction of the balloon device.

It would be desirable that the balloon structure itself should include an increase in the shaft diameter at the proximal end of the shaft to improve recovery of plaque material, and to prevent back flow.

It would also be desirable to provide a hemostasis valve with a screw-on lock which prevents back movement of a dilator in the introducer shaft during an insertion procedure. This is not provided in the Horita application, supra.

### THE INVENTION

According to the invention there is provided an apparatus and method for producing an improved construction for a balloon catheter insertion introducer shaft comprising a polymeric balloon material and a bonding agent for the balloon and shaft comprising a liquid or viscous polymeric oligomer of the same or similar polymeric composition as the balloon. The oligomer may include curable bonds such as a primer, or the oligomer may be admixed with a primer or curing agent. Typically, the oligomer is bonded to a shaft made of the same polymeric material as the balloon in order to achieve maximum bonding efficiency.

Various balloon materials may be employed such as neoprene, natural rubber latex, polyisoprene, silicone, nylon, polyethylene, polypropylene, polyurethane, polyester and PVC.

Shaft materials can include corresponding polymer materials; PEBAX® (polyether block amide); and, other polymers which can effectively bond with a given balloon polymer. Besides oligomers, specific adhesives for these balloon polymers are also disclosed in MODERN PLASTICS ENCYCLOPEDIA, 1981-1982, October 1981 Volume 58, Number 10a, pages 432, et seq, and published by McGraw Hill. Other bonding techniques are described on pages 434-440; and, electrostatic/corona discharge may also be used to provide suitable bonding characteristics. The entire MODERN PLASTICS ENCYCLOPEDIA, supra, is incorporated herein by reference.

Although silicone and PEBAX® are dissimilar polymers, their combination represents the preferred embodiment, particularly since use of a silicone balloon provides suitable characteristics such as ease of inflation, hardness, and low friction properties. This makes silicone balloons easy to insert and retract from the lesion or plaque and the vein or artery. Additionally, silicone balloons do not tend to rupture when inflated, and the compliant, conforming and elasticity of silicone balloons enables inflation of the balloons to be safely made without over-expansion, which could lead to a blow-out of a vein/artery. Also, if a silicone balloon rupture occurs, it will not fragment, which is the case with other balloon materials. Moreover, in a compliant system, leakage of blood and/or emboli and debris is virtually eliminated.

By contrast, current non-compliant balloons do not respond well to artery pressure, and hence this property could result in suction and artery rupture. Also, this non-compliant property does not seal off leakage of blood or debris/emboli.

When bonding a silicone balloon to PEBAX®, use of a suitable coupling agent as a primer is preferable. Typically, the cure bonding is at an elevated temperature over a suitable time period. PEBAX® polymers are sold by AUTOCHEM who publish catalogues of properties and uses of this material.

Bonding techniques for silicone polymers are described in U.S. Pat. Nos. 5,762,996 and 5,795,332 to the inventors herein and incorporated by reference hereto. As disclosed in U.S. patent application Ser. No. 11/079,130, filed Mar. 12, 2005 and incorporated herein by reference, the viscosity of the silicone and solvent (e.g., isopropanol, xylene, etc.), is adjusted prior to manufacturing the catheter, to control catheter softness.

The silicone coating which forms the balloon catheter is made thicker and also coated well behind and along the shaft, so that blood carrying emboli material and dye will not leak or back flow along the shaft or balloon. Suitable coating thicknesses vary from about 0.012″-0.20″. Suitable balloon thicknesses may vary from about 0.0035″-0.016″, and a deflated balloon diameter may vary between about 0.052″-0.158″, i.e. about 4-12 french.

### BRIEF DESCRIPTION OF THE DRAWINGS

FIG. 1 is an external side elevation view, partly in axial section, showing the prior art device described in the Japanese patent, supra;

FIG. 2 is an external side elevation view, partly in axial section, showing the device of the present invention;

FIG. 2A is a top plan view of FIG. 2, taken along lines 2-2 of FIG. 2.

FIG. 3 is an enlargement of the circled area of FIG. 2; and,

FIG. 4 is a diagrammatic view showing the balloon and introducer shaft positioned in an artery or vein during a stenosis dilation procedure, and also as described in the Horita application, supra.

### DESCRIPTION OF THE PREFERRED EMBODIMENTS

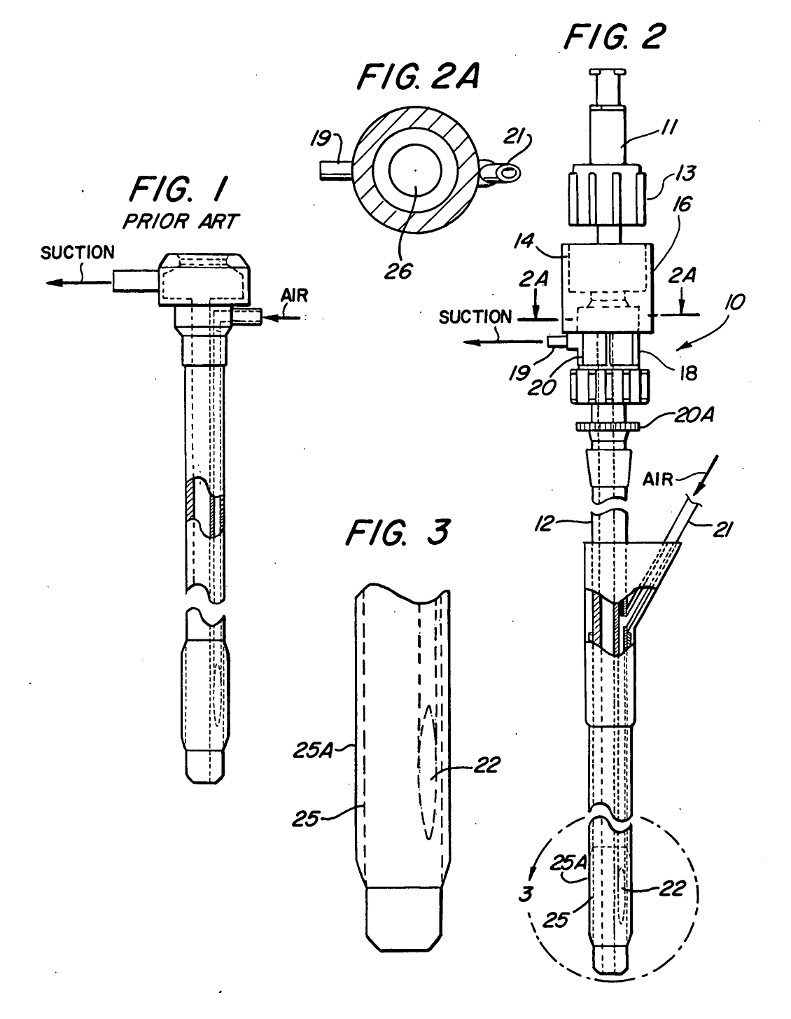

The percutaneous translumen introducer (PTI) 10 is shown in FIG. 2 and includes a dilator 11 for an introducer shaft 12 which is secured in a lock nut 13 that is mounted within a locking connector 14, and these components are fitted into a sleeve 16. The lock nut 13 is threadably engaged with a hemostasis valve 18, and hence this screw-on lock with the hemostasis valve prevents back movement of the dilator 11 within The introducer shaft 12.

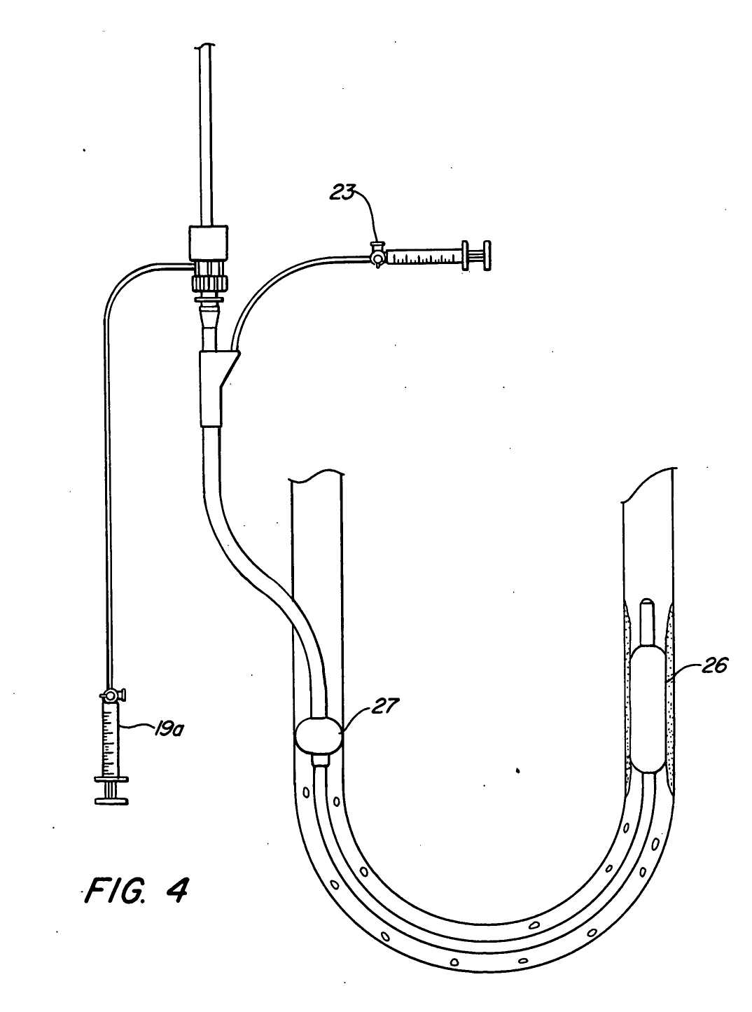

The hemostasis valve 18 provides a suction port 19, suction syringe 19A (FIG. 4), and a three-way valve 20 for removal of emboli material. A luer connector 20A connects the hemostasis valve with the introducer 10. Downstream from the hemostasis valve is a balloon inflation/deflation line 21 leading to a balloon inflation port 22, and a three-way stopcock 23 (FIG. 4).

A balloon catheter 25 is mounted on the introducer shaft for initially inserting into a patient's vein and/or artery. As shown in FIG. 4, one lumen forms a working channel 26 for introduction of a guide wire. This guide wire enables subsequent introduction of a catheter to dilate a lesion or plaque. The other lumen forms an occlusion balloon 27 to prevent back movement of both dye and released emboli from the lesion. Back movement of emboli could cause an infarction that is potentially dangerous to a patient. Typical introducers useful in conjunction with balloon catheters include over-the-wire and embolectomy catheters.

As indicated, supra, the silicone coating 25A which forms the balloon catheter is made thicker and also coated well behind and along the shaft so that blood carrying emboli material and dye will not leak or back flow along the shaft or balloon. Alternatively, a double thickness (e.g., sleeve) may be utilized.

The three-way stopcock 23 is provided to inject a suitable amount of inflation air or fluid (e.g., saline) to a balloon, and to release pressure and deflate the occlusion balloon upon completion of a lesion or plaque dilation procedure.

Prior to dilation of a lesion or plaque, their contours may be determined such as by Quantitative Coronary Angiography (QCA), as described in the article, “EVALUATION OF CENTER-LINE EXTRACTION ALGORITHM IN QUANTITATIVE CORONARY ANGIOGRAPHY”, by H. Greenspan, et al, IEEE Transactions on Medical Imaging, Vol. 20, No. 9, September 2001. A suitable software program, CATHLAB, may be utilized for this purpose.

During dilation of the lesion or plaque, the procedure as illustrated in FIG. 4 should take place within about ninety (90) seconds, otherwise the blockage of blood flow could result in serious injury to vital organs of a patient due to lack of blood carrying oxygen. Obviously, ease of insertion and retraction of a silicone balloon expedites the procedure.

Following suitable dilation of the lesion or plaque, and removal of dye and emboli material from the suction port 19 (using the suction syringe 19A), the occlusion balloon 27 is deflated and removed, and the procedure is concluded.

1. A percutaneous translumen introducer for a balloon catheter, comprising:
a.) an introducer holder;
b.) a hemostasis valve providing suction means for removal of dye and embolus material arising from dilation of a lesion of a plaque site; and,
c.) means for securing the introducer holder to the hemostasis valve to prevent backward movement of the introducer holder during movement of the holder.

2. The percutaneous translumen introducer and associated balloon catheter of claim 1, comprising:
a.) an introducer holder;
b.) a dilator holder for the introducer;
c.) a lock nut for securing the dilator;
d.) a locking connector for securing the lock nut;
e.) a sleeve for engaging the locking connector;
f.) a hemostasis valve engaging the sleeve, thereby preventing backward movement of the introducer during advancement and use; and,
g.) locking means for securing the hemostasis valve to the introducer.

3. A percutaneous translumen introducer, comprising a balloon introducer; an insertion introducer shaft; a silicone dilation balloon mounted on the introducer shaft for insertion into a vein or artery following the introducer, the dilation balloon being coated along the introducer shaft and extending backwards along the shaft; the silicone dilation balloon being constructed and adapted to dilate a lesion or plaque formation in the vein or artery; an occlusion balloon introducer catheter, an occlusion balloon upon inflation being adapted to prevent back flow of emboli released from the dilation of the lesion or plaque; inflation and deflation means for the dilation balloon; and, means for inflation and deflation of the dilation and occlusion balloons, and for removal of released emboli.

4. The percutaneous translumen introducer of claim 3, in which the coating of the dilation balloon backwards along the introducer shaft varies from about 0.012″-0.20″ in thickness.

5. The percutaneous translumen introducer of claim 3, in which the dilation balloon is about 0.0035″-0.016″ thick.

6. The percutaneous translumen introducer of claim 3, in which the coating of the dilation balloon backwards along the introducer shaft is about 0.012″-0.20″ thick, and the dilation balloon is about 0.0035″-0.016″ thick.

7. The percutaneous translumen introducer of claim 3, in which the coating of the dilation balloon backwards along the introducer shaft is about 0.012″-0.20″ thick, the dilation balloon is about 0.0035″-0.016″ thick, and the deflated balloon diameter is about 0.052″-0.158″.

8. A method for performing a percutaneous translumen procedure, comprising inserting an introducer for a vein or artery; providing an introducer shaft; bonding a silicone dilation balloon onto the introducer shaft; following the introducer into the vein or artery with the introducer shaft and silicone dilation balloon; inflating the dilation balloon to dilate a lesion, plaque or the like; inserting and inflating an occlusion balloon introducer catheter, thereby preventing back flow of emboli released from the dilation or plaque; removing released emboli collected at the occlusion balloon; and, deflating and removing the dilation and occlusion balloons.

9. The method of claim 8, in which a silicone coating, provides a double thickness, sleeve or the like extending from the silicone dilation balloon backwards along the occlusion balloon introducer catheter, thereby aiding in the prevention of back flow of emboli released from dilation of a lesion or plaque.

10. The method of claim 9, in which the silicone coating varies in thickness from about 0.012″-0.20″.

11. A method for coating a balloon onto a catheter introducer shaft comprising the steps of bonding the balloon to the introducer shaft with a bonding agent comprising a low molecular weight oligomer having the same or suitable structure as the polymer of the introducer shaft.

12. The method of claim 11, in which the balloon and shaft are selected from the class consisting of neoprene, natural rubber latex, polyisoprene, silicone, nylon, polyethylene, polyester, polypropylene and, polyurethane.

13. The method of claim 11, in which the balloon is a silicone polymer and the shaft is a polyether block amide.

14. The method of claim 13, in which the balloon is about 0.0035″-0.016″ in thickness, and a deflated balloon diameter is about 0.052″-0.158″.
